(19) United States
(12) Patent Application Publication
(10) Pub. No.: US 2007/0178147 A1
(43) Pub. Date: Aug. 2, 2007

(54) Liposomal compositions

(76) Inventors: Narendra R. Desai, Ridgefield, CT (US); Hemlata R. Dalal, Montville, NJ (US); Richard W. Saunders, Palisades, NY (US)

Correspondence Address: FISH & RICHARDSON P.C., P.O BOX 1022, MINNEAPOLIS, MN, 55440-1022, US

(21) Appl. No.: 11/636,378

(22) Filed: Dec. 8, 2006

Related U.S. Application Data
(60) Provisional application No. 60/748,686, filed on Dec. 8, 2005.

(51) Int. Cl. A61K 9/127

(52) U.S. Cl. 424/450

(57) ABSTRACT

This invention relates generally to liposomal pharmaceutical compositions and related methods.

### CROSS REFERENCE TO RELATED APPLICATIONS

This application claims the benefit of U.S. Provisional Application No. 60/748,686, filed on Dec. 8, 2005, which is incorporated herein by reference in its entirety.

### TECHNICAL FIELD

This invention relates generally to liposomal pharmaceutical compositions and related methods.

### BACKGROUND

In some instances, in the treatment of humans or animals with drugs, it may be necessary to administer the drug by the intravenous route. Intravenous administration is among the most rapid and direct means of drug delivery. However, local intravenous injection site adverse reactions can occur as a result of (a) thermodynamically driven local precipitation of the drug in venous blood (e.g., local thrombophlebitis, chemical phlebitis); (b) preferential binding of the drug with the injection site tissue causing relatively high local accumulation of the drug, or (c) a needle damaged vein, which can lead to extravasation followed by attack of the exposed tissue by the drug.

### SUMMARY

This invention relates generally to liposome-forming pharmaceutical compositions, and water-based formulations thereof, which contain one or more hydrophobic therapeutic agents (e.g., drugs). Such formulations preferably can be used to achieve pre- and post-delivery (e.g., pre- and post-injection) solubilization of a hydrophobic therapeutic agent when administered (e.g., intravenously administered) to a subject (e.g., a subject in need thereof) in aqueous vehicles that lack a co-solvent(s) (e.g., an organic solvent) that is miscible with the hydrophobic therapeutic agent.

In one aspect, this invention relates to a lyophilized liposomal composition, which includes: (i) a hydrophobic therapeutic agent; (ii) a first component; and (iii) a second component; in which, when the composition is contacted with water, the first component and the second component interact to form a substantially homogeneous liposomal solution of the hydrophobic therapeutic agent.

In another aspect, this invention relates to a process for preparing a lyophilized liposomal composition, which includes: (i) combining a hydrophobic therapeutic agent, a first component, and a second component in an organic solvent to form a first combination; (ii) combining the first combination with a water phase to form a second combination; (iii) removing the organic solvent from the second combination to form a third combination (e.g., removing some or substantially all of the organic solvent, e.g., by distillation; evaporation under reduced pressure (e.g., aspirator pressure or low vacuum, e.g., from about 1 mmHg to about 50 mmHg); or tangential flow filtration); and (iv) lyophilizing the third combination, thereby preparing the lyophilized liposomal composition. In embodiments, the methods can be used for the large scale manufacture of hydrophobic drugs (e.g., sterile hydrophobic drugs) and can provide a relatively simple “one-pot” method for the manufacturing of sterile pharmaceutical liposomal products.

In a further aspect, this invention relates to a process for preparing a lyophilized liposomal composition, which includes: (i) combining a hydrophobic therapeutic agent, a first component, and a second component in an organic solvent to form a first combination; (ii) removing the organic solvent from the first combination to form a second combination (e.g., removing some or substantially all of the organic solvent to form, e.g., a thin film); (iii) combining the second combination with a water phase to form a third combination; and (iv) lyophilizing the third combination, thereby preparing the lyophilized liposomal composition.

In one aspect, this invention relates to a substantially homogeneous liposomal formulation, which includes: (i) a hydrophobic therapeutic agent; (ii) a first component; (iii) a second component; and (iv) water.

Embodiments can include one or more of the following features.

The hydrophobic therapeutic agent can have a log P value of from about 1.0 to about 5.0 (e.g., from about 2.0 to about 5.0, from about 3.0 to about 5.0, from about 4.0 to about 5.0).

The composition can include from about 20 weight percent to about 40 weight percent of the first component and the second component.

The weight per cent ratio of the second component to the first component can be from about 1 to about 7 (e.g., from about 1 to about 5, from about 2 to about 5, from about 1 to about 3, from about 2 to about 3). The weight per cent ratio of the second component to the first component can be from about 2.2 to about 2.7. The weight per cent ratio of the second component to the first component can be from about 4 to about 5 (e.g., from about 4.2 to about 4.8, e.g., 4.5).

The number of moles of the first component can be less than the number of moles of the hydrophobic therapeutic agent. For example, the (ratio of the first component): hydrophobic therapeutic agent can be (from about 0.10 to about 0.95):1; e.g., (from about 0.50 to about 0.95):1; e.g., about (0.75):1.

The number of moles of the first component can be about the same as the number of moles of the hydrophobic therapeutic agent.

The number of moles of the first component can be from about 1.5 to about 6 times greater than the number of moles of the hydrophobic therapeutic agent.

The number of moles of the second component can be from about 2 to about 15 times greater than the number of moles of the hydrophobic therapeutic agent.

The number of moles of the first component can be less than the number of moles of the hydrophobic therapeutic agent, and the number of moles of the second component can be from about 2 to about 15 times greater than the number of moles of the hydrophobic therapeutic agent.

The number of moles of the first component can be about the same as the number of moles of the hydrophobic therapeutic agent, and the number of moles of the second component can be from about 2 to about 15 times greater than the number of moles of the hydrophobic therapeutic agent.

The number of moles of the first component can be from about 1.5 to about 6 times greater than the number of moles of the hydrophobic therapeutic agent, and the number of moles of the second component can be from about 2 to about 15 times greater than the number of moles of the hydrophobic therapeutic agent.

For example, the molar ratio of the hydrophobic therapeutic agent:first component:second component can be about:

1:0.75:3
1:1:5
1:3:7
1:4:11

The weight per cent ratio of the first component and the second component to the hydrophobic therapeutic agent can be from about 2 to about 50 (e.g., from about 10 to about 50).

The weight per cent ratio of the first component and the second component to the hydrophobic therapeutic agent can be from about 15 to about 25.

Each of the first component and the second component can be, independently, a natural lecithin or a phospholipid (e.g., derived from egg, soy, or vegetable phospholipids or synthetic phospholipids).

The first component can be phosphatidyl glycerol and the second component can be phosphatidyl choline (e.g., derived from egg, soy, or vegetable phospholipids or synthetic phospholipids). For example, the first component can be egg phosphatidyl glycerol, and the second component can be soy phosphatidyl choline.

The composition can include from about 0.05 weight percent to about 10 weight percent of the hydrophobic therapeutic agent.

The composition can further include a cryoprotectant (e.g., a sugar, e.g., lactose).

The composition can further include an anti-oxidant. In certain embodiments, the composition can include two anti-oxidants (e.g., BHT and ascorbyl palmitate). In certain embodiments, the composition can include more than two anti-oxidants.

The composition can further include a cryoprotectant, a first anti-oxidant, and a second anti-oxidant.

The cryoprotectant can be lactose, the first anti-oxidant can be BHT, and the second anti-oxidant can be ascorbyl palmitate.

The hydrophobic therapeutic agent can have a water solubility of from about 5 nanograms/mL to about 5 milligrams/mL (e.g., from about 5 nanograms/mL to about 2 milligrams/mL).

The hydrophobic therapeutic agent can have a molecular weight of from about 100 Daltons to about 1,000 Daltons.

The hydrophobic therapeutic agent can lack ionizable groups.

The hydrophobic therapeutic agent can further include an acidic group having a pKa of from about 2 to about 11.

The hydrophobic therapeutic agent can further include a basic group, wherein the pKa of the basic group's conjugate acid can be from about 3 to about 12.

The hydrophobic therapeutic agent can further include one or more acidic groups having a pKa of from about 2 to about 11 and one or more basic group, wherein the pKa of the basic group's conjugate acid is from about 3 to about 12. For example, the hydrophobic therapeutic agent can be a zwitterion.

The hydrophobic therapeutic agent can be a crystalline solid.

The hydrophobic therapeutic agent can be a hydrophobic liquid (e.g., an oil).

The hydrophobic therapeutic agent can further include two rings, wherein each ring can be, independently, an aromatic ring or a heteroaromatic ring.

The hydrophobic therapeutic agent can further include a condensed bicyclic, tricyclic or polycyclic ring system (e.g., of synthetic or natural origin).

The hydrophobic therapeutic agent can be a water insoluble fungal antibiotic or complex macrocycle of synthetic, semi-synthetic, or natural origin.

The organic solvent can be ethanol.

The water phase can further include a cryoprotectant (e.g., lactose).

The first combination can further include an anti-oxidant.

The second combination can be a liposomal solution.

The process can further include the step of reducing the average particle size distribution of the liposomes. For example, the process can further include the step of reducing the particle size distribution of the (e.g., coarse) liposomes to a final particle size distribution of from about 5,000 nm to about 20 nm, e.g., from about 5,000 nm to about 50 (i.e., the particle size distribution of the liposomes after performing this particle size reduction step is, for example, from about 5,000 nm to about 20 nm). For example, the process can further include the step of reducing the particle size distribution of the liposomes to about 200 nanometers (nm) or lower (e.g., at most about 200 nm, less than 200 nm). For example, the process can further include the step of reducing the particle size distribution of the liposomes to from about 200 nm to about 20 nm, e.g., from about 200 nm to about 50 nm (i.e., the particle size distribution of the liposomes after performing this particle size reduction step is, for example, from about 200 nm to about 20 nm).

Step (iii) can include performing a tangential flow filtration. The organic solvent can be ethanol.

The formulation can include at least about 80 weight/volume per cent of water.

The formulation can further include a cryoprotectant, a first anti-oxidant, and a second anti-oxidant.

The formulation can include from about 2 mg/mL to about 10 mg/mL (e.g., from about 2 mg/mL to about 8 mg/mL, e.g., about 2 mg/mL) of the hydrophobic therapeutic agent.

The formulation can be an intravenous formulation or parenteral formulation for administration to a human or animal subject.

The formulation can be prepared by contacting the lyophilized lipsomal compositions described herein with water.

The liposomes can have an average particle size distribution of at most about 5,000 nm.

The liposomes can have an average particle size distribution of from about 20 nm to about 300 nm, e.g., from about 50 nm to about 300 (e.g., about 200 nm).

The formulation can be capable of being diluted indefinitely with water without precipitation of the hydrophobic therapeutic agent.

The formulation can be fast breaking. In embodiments, the formulation (liposome) can rapidly release the hydrophobic therapeutic agent into the bloodstream to associate with, e.g., red blood cell (RBC), lipoproteins, HSA or WBC in blood upon in vivo administration. It is believed that this reduces the likelihood of the hydrophobic therapeutic agent from being accumulated in non-target tissues such as the liver, where conventional liposomes otherwise have a tendency to concentrate. While not wishing to be bound by theory, it is believed that the “fast breaking” nature of the liposomes of the liposomal compositions and formulations described herein can be due to the manner in which the hydrophobic therapeutic agent associates with the lipid bilayer of the liposomes.

As used herein, the term “hydrophobic therapeutic agent” refers to a bioactive moiety that is sparingly soluble, slightly soluble, very slightly soluble, practically insoluble, or insoluble in water, which when administered to a subject (e.g., a human or animal subject) in an amount of from about 0.01 mg/Kg to about 1000 mg/Kg, (e.g., from about 0.01 mg/Kg to about 500 mg/kg, from about 0.1 mg/Kg to about 250 mg/Kg, from about 1 mg/Kg to about 100 mg/Kg, from about 1 mg/Kg to about 10 mg/kg) confers a therapeutic, biological, or pharmacological effect (e.g., treats, controls, ameliorates, prevents, delays the onset of, or reduces the risk of developing one or more diseases, disorders, or conditions or symptoms thereof) on the treated subject. The therapeutic effect may be objective (i.e., measurable by some test or marker) or subjective (i.e., subject gives an indication of or feels an effect) and can be local or systemic.

As used herein, the terms “sparingly soluble, slightly soluble, very slightly soluble, practically insoluble, or insoluble” correspond in meaning to the United States Pharmacopeia (USP) general terms for approximate solubility expression (see, e.g., DeLuca and Boylan in Pharmaceutical Dosage Forms: Parenteral Medications, vol. 1, Avis, K. E., Lachman, L. and Lieberman, H. A., eds; Marcel Dekkar: 1084, pages 141-142:

| | Relative amount of |
|---|---|
| | solvent to dissolve 1 |
| USP term | part of solute |
| sparingly soluble | 30-100 |
| Slightly soluble | 100-1,000 |
| very slightly soluble | 1,000-10,000 |
| practically insoluble, or insoluble | >10,000 |

By way of example, a sparingly soluble hydrophobic therapeutic agent is one in which from about 30 to about 100 parts of water is needed to dissolve about 1 part of the hydrophobic therapeutic agent. Similarly, a slightly soluble hydrophobic therapeutic agent is one in which from about 100 to about 1,000 parts of water is needed to dissolve about 1 part of the hydrophobic therapeutic agent; a very slightly soluble hydrophobic therapeutic agent is one in which from about 1,000 to about 10,000 parts of water is needed to dissolve about 1 part of the hydrophobic therapeutic agent; and a practically insoluble, or insoluble hydrophobic therapeutic agent is one in which more than about 10,000 parts of water is needed to dissolve about 1 part of the hydrophobic therapeutic agent.

“Bioactive moieties” can include, for example, a drug approved by a regulatory agency (e.g., the United States (US) Food and Drug Administration, Department of Agriculture, or their non-US equivalents), a drug candidate under review by a regulatory agency (e.g., a phase 0, 1, 2, or 3 drug candidate, e.g. a drug candidate undergoing clinical trials), or a compound identified as a lead compound by a public or private research entity on the basis of the results of conventional screening method or in vitro or in vivo assay. The term “hydrophobic therapeutic agent” excludes, for example, the porphyrin photosensitizers described in U.S. Pat. Nos. 6,074,666 and 6,890,555 and 7,135,193B2 (e.g., benzoporphyrin derivatives (BPD), e.g., BPD mono acid (BPDMA).

As used herein, the term “liposome” refers to a completely closed lipid bilayer membrane containing an entrapped aqueous volume, which is formed spontaneously on addition of an aqueous solution to a dry phospholipid film (e.g., obtained by rotary evaporation as described herein) or a phospholipid solution (e.g., obtained by tangential flow filtration as described herein). Liposomes include unilamellar vesicles having a single membrane bilayer or multilamellar vesicles having multiple membrane bilayers, each separated from the next by an aqueous layer. The bilayer includes two lipid monolayers having a hydrophobic “tail” region and a hydrophilic “head” region. While not wishing to be bound by theory, the structure of the membrane bilayer is such that the hydrophobic (non polar) “tails” of the lipid monolayers orient towards the center of the bilayer while the hydrophilic “heads” orient toward the aqueous phase.

As used herein, the term “liposomal solution” refers generally to aqueous or aqueous/organic solvent dispersions of hydrophobic therapeutic agent-encapsulated liposomes of any average particle size distribution.

As used herein, the terms “substantially homogeneous liposomal solution of the hydrophobic therapeutic agent” or “substantially homogeneous liposomal formulation of the hydrophobic therapeutic agent” refer to a homogeneous, aqueous dispersion of hydrophobic therapeutic agent-encapsulated liposomes, in which the liposomes have an average particle size distribution of from about 20 nm to about 5,000 nm (e.g., from about 50 nm to about 5,000 nm, e.g., at most about 200 nm, less than 200 nm). The average particle size distribution of liposomal solutions described herein can be determined by conventional methods in the art (e.g., light scattering, e.g., dynamic laser light scattering using, e.g., submicron particle measuring systems such as those available from Nicomp or Malvern).

As used herein, the term “subject” refers to organisms, which include mice, rats, cows, sheep, pigs, rabbits, goats, and horses, monkeys, dogs, cats, and preferably humans.

The details of one or more embodiments of the invention are set forth in the description below. Other features and advantages of the invention will be apparent from the description and from the claims.

### DETAILED DESCRIPTION

In some embodiments, a lyophilized liposomal composition can include one or more hydrophobic therapeutic agents, a first component, a second component, a cryoprotectant, a first anti-oxidant, and a second anti-oxidant.

Hydrophobic Therapeutic Agents

Preferred hydrophobic therapeutic agents can have one or more of the following physical, structural or stereochemical or chemical attributes.

(1) The hydrophobic therapeutic agent can have an octanol/water partition coefficient (log P) value of from about 1.0 to about 5.0 (e.g., 1.0, 1. 1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0).

For ease of exposition, it is understood that any recitation of ranges (e.g., log P of from about 1.0 to 5.0) or subranges of a particular range (e.g., log P of from about 1.0 to 1.5) expressly includes each of the individual values that fall within the recited range, including the upper and lower limits of the recited range.

In certain embodiments, the hydrophobic therapeutic agent can have a log P of from about 1.0 to about 5.0 (e.g., from about 1.0 to about 4.5, from about 1.0 to about 4.0, from about 1.0 to about 3.5, from about 1.0 to about 3.0, from about 1.0 to about 2.5, from about 1.0 to about 2.0, from about 1.0 to about 1.5).

In certain embodiments, the hydrophobic therapeutic agent can have a log P of from about 2.0 to about 5.0 (e.g., from about 2.0 to about 4.5, from about 2.0 to about 4.0, from about 2.0 to about 3.5, from about 2.0 to about 3.0, from about 2.0 to about 2.5).

In certain embodiments, the hydrophobic therapeutic agent can have a log P of from about 2.5 to about 5.0 (e.g., from about 2.5 to about 4.5, from about 2.5 to about 4.0, from about 2.5 to about 3.5, from about 2.5 to about 3.0).

In certain embodiments, the hydrophobic therapeutic agent can have a log P of from about 3.0 to about 5.0 (e.g., from about 3.0 to about 4.5, from about 3.0 to about 4.0, from about 3.0 to about 3.5).

In certain embodiments, the hydrophobic therapeutic agent can have a log P of from about 3.5 to about 5.0 (e.g., from about 3.5 to about 4.5, from about 3.5 to about 4.0).

In certain embodiments, the hydrophobic therapeutic agent can have a log P of from about 4.0 to about 5.0 (e.g., from about 4.0 to about 4.5).

In certain embodiments, the hydrophobic therapeutic agent can have a log P of from about 4.5 to about 5.0.

(2) The hydrophobic therapeutic agent can have a water solubility of from about 5 nanograms/mL to about 5 milligrams/mL (e.g., from about 5 nanograms/mL to about 4 milligrams/mL, from about 5 nanograms/mL to about 3 milligrams/mL, from about 5 nanograms/mL to about 2 milligrams/mL, from about 5 nanograms/mL to about 1 milligram/mL, from about 5 nanograms/mL to about 0.5 milligrams/mL, from about 5 nanograms/mL to about 0.25 milligrams/mL, from about 5 nanograms/mL to about 0.1 milligrams/mL). In certain embodiments, the hydrophobic therapeutic agent can have a water solubility of from about 5 nanograms/mL to about 2 milligrams/mL.

(3) The hydrophobic therapeutic agent can have a molecular weight of from about 100 Daltons (D) to about 2000 D (e.g., from about 100 D to about 1500 D, from about 100 D to about 1000 D, from about 200 D to about 800 D).

(4) The hydrophobic therapeutic agent can include an acidic group (i.e., a moiety containing one or more dissociable protons), in which the pKₐ (relative to water) of the dissociable proton(s) is(are) from about 2 to about 11 (e.g., from about 2 to about 10, from about 2 to about 7, from about 4 to about 11, from about 4 to about 10, from about 4 to about 7) pKₐ units.

(5) The hydrophobic therapeutic agent can include a basic group, in which the pKₐ (relative to water) of the basic group's conjugate acid is from about 1.5 to about 12 (e.g., about 3 to about 12, about 5 to about 12).

(6) The hydrophobic therapeutic agent can include an acidic group in which the pKₐ (relative to water) of all dissociable proton(s) is(are) greater than about 11 and/or a basic group, in which the pKa (relative to water) of the basic group's conjugate acid is less than about 1.5.

(7) The hydrophobic therapeutic agent can include any combination or number of groups delineated in (4), (5), and (6). For example, the hydrophobic therapeutic agent can include one or more acidic groups as described herein and one or more basic groups as described herein. In some embodiments, the hydrophobic therapeutic agent can be a zwitterion or dipolar ion (a neutral molecule having oppositely charged moieties, e.g., a moiety that is the product of the reaction (proton exchange) between an acidic group (e.g., —COOH, —P(O)(OH)₂, or —SO₃H), a basic group (e.g., —NH₂, secondary or tertiary amino) that are both present on the same molecule), and a zwitterionic groups (e.g. amino acids, peptides and proteins).

(8) The hydrophobic therapeutic agent can include only one or more groups delineated in (4).

(9) The hydrophobic therapeutic agent can include one or more asymmetric centers and thus be present together with one or more isomeric forms of the hydrophobic therapeutic agent in the compositions and formulations described herein. As such, the compositions and formulations described herein can include racemates and racemic mixtures, single enantiomers, individual diastereomers and diastereomeric mixtures of a hydrophobic therapeutic agent. Similarly, the hydrophobic therapeutic agent can also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, the compositions and formulations described herein can include cis/trans and E/Z isomer and/or rotational isomer mixtures of the hydrophobic therapeutic agent. The compositions and formulations described herein can also include tautomeric mixtures of the hydrophobic therapeutic agent.

(10) The physical form of the hydrophobic therapeutic agent can be selected as desired (e.g., based on stability considerations or ease of isolation and handling). For example, the hydrophobic therapeutic agent can be a crystalline solid, a polymorph, an amorphous solid, or a hydrophobic liquid (e.g., an oil).

(11) The hydrophobic therapeutic agent can include one or more moieties that are known in the art to confer hydrophobicity to a chemical compound (e.g., C₁₋₂₀ (e.g., C₅₋₁₈) alkyl, C₂₋₂₀ (e.g., C₅₋₁₈) alkenyl, or C₂-C₂₀ (e.g., C₅₋₁₈) alkynyl straight or branched chains; or C₃-C₂₀ saturated or partially saturated carbocyclic rings; or aromatic or heteroaromatic rings containing from 5-18 atoms). In certain embodiments, the hydrophobic therapeutic agent can include two rings, each of which can be independently of one another, an aromatic ring or a heteroaromatic ring. The two rings can be in conjugation with respect to one another either through connection via a single bond or by forming part of a condensed (fused) bicyclic, tricyclic or polycyclic ring systems (e.g., of synthetic or natural origin).

Hydrophobic therapeutic agents can include, but are not limited to, Src kinase inhibitors, cardiomyocyte gap junction modifiers, anti-inflammatory drugs (e.g., steroidal and nonsteroidal); antibacterials; antiprotozoals; antifungals; coronary vasodilators; calcium channel blockers; bronchodilators; enzyme inhibitors such as collagenase inhibitors, protease inhibitors, elastase inhibitors, lipoxygenase inhibitors, and angiotensin converting enzyme inhibitors; other antihypertensives; leukotriene antagonists; anti-ulceratives such as H2 antagonists; steroidal hormones; antivirals and/or immunomodulators; local anesthetics; cardiotonics; antitussives; antihistamines; narcotic analgesics; peptide hormones; sex hormones; cardioactive products such as atriopeptides; proteinaceous products; antinauseants; anticonvulsants; immunosuppressives; psychotherapeutics; sedatives; anticoagulants; analgesics; antimigraine agents; antiarrhythmic agents; antiemetics; anticancer agents; neurologic agents such as anxiolytic drugs; hemostatics; anti-obesity agents; antimicrobial agents; serotonin pathway modulators; cyclic nucleotide pathway agents; catecholamine modulators; endothelin receptor antagonists; nitric oxide donors/releasing molecules; ATII-receptor antagonists; platelet adhesion inhibitors; platelet aggregation inhibitors; coagulation pathway modulators; cyclooxygenase pathway inhibitors; lipoxygenase pathway inhibitors; antagonists of E- and P-selectins; inhibitors of VCAM-1 and ICAM-1 interactions; prostaglandins and analogs thereof; macrophage activation preventers; HMG-CoA reductase inhibitors; agents affecting various growth factors (including FGF pathway agents, PDGF receptor antagonists, IGF pathway agents, TGF-β pathway agents, EGF pathway agents, TNF-α pathway agents, Thromboxane A2 [TXA2] pathway modulators, and protein tyrosine kinase inhibitors); MMP pathway inhibitors; cell motility inhibitors; anti-inflammatory agents; antiproliferative/antineoplastic agents;

matrix deposition/organization pathway inhibitors; endothelialization facilitators; blood rheology modulators; as well as integrins, chemokines, cytokines and growth factors.

Preferred therapeutic agents include, for example, water insoluble fungal antibiotics and complex natural, synthetic, or semi-synthetic macrocycles derived from plant, marine or animal sources (e.g., paclitaxel, docetaxel, rapamycin). Preferred therapeutic agents can include those obtained from terrestrial sources, such as clays, dirt, soil, or earth (e.g., from surface layers of the earth; mines; dried river, lake, lagoon beds).

Hydrophobic therapeutic agents also include genetic therapeutic agents and proteins, such as ribozymes, anti-sense polynucelotides and polynucleotides coding for a specific product (including recombinant nucleic acids) such as genomic DNA, cDNA, or RNA. The polynucleotide can be provided in “naked” form or in connection with vector systems that enhances uptake and expression of polynucleotides. These can include DNA compacting agents, non-infectious vectors and viral vectors such as viruses and virus-like particles (i.e., synthetic particles made to act like viruses). The vector may further have attached peptide targeting sequences, antisense nucleic acids, and DNA chimeras which include gene sequences encoding for ferry proteins such as membrane translocating sequences (“MTS”) and herpes simplex virus-1 (“VP22”).

In general, lyophilized liposomal compositions can include from about 0.05 weight percent to about 10 weight percent (relative to the total weight of the composition) of the hydrophobic therapeutic agent (e.g., from about 0.500 weight percent to about 2.500 weight percent, from about 1.000 weight percent to about 2.000 weight percent).

Components

The first and second components are moieties that interact to form the liposome lipid bilayer when the lyophilized liposomal compositions are contacted with water.

In general, a high crystal lattice energy can lead to a high melting point and low aqueous solubility. Crystal lattice energy can be increased, for example, by π-stacking interactions (e.g., stacking of aromatic rings). This intermolecular stacking is believed to arise from an asymmetry in polarity in different regions of the molecule which complement each other and is believed to contribute to the water insolubility of some hydrophobic therapeutic agents (HTA) having multiple π systems. While not wishing to be bound by theory, it is believed that if this π -π electronic interaction can be reduced by inserting molecules with anionic head groups and hydrophobic tails, then the lattice energy contribution can be lowered and the aqueous solubility of the HTA can be increased. It is further believed that interaction (e.g., complexation) of a hydrophobic therapeutic agent (e.g., a hydrophobic therapeutic agent having multiple π systems or any hydrophobic therapeutic agent having a high associated crystal lattice energy) with one or more low melting hydrophobic phospholipids (e.g., those having a medium length fatty acid chain and/or an fatty acid chain containing one or more unsaturations) and/or with polymers with anionic charge or electron donating capability can lower the melting point of the hydrotherapeutic agent in, e.g., the complex. When, for example, hydrophobic therapeutic agent:lipid complexes are exposed to water, organized assemblies such as liposomes or micelles can be formed due to balancing of the hydrophobic and electrostatic interactions. As a result, operational aqueous solubility can be achieved.

Thus, in some embodiments, the first and second component can each, independently, include one or more fatty acid chains having a medium chain length and/or one or more degrees of unsaturation. While not wishing to be bound by theory, it believed that fatty acids having one or both of these properties can have decreased melting points, and their presence in the components of the liposomal compositions and formulations described herein can increase the degree of incorporation of lipophilic or hydrophobic material in the bilayer. The presence of such fatty acid chains on the components can also increase the bilayer fluidity of the resultant liposomes and allow penetration of the resultant liposomes by blood proteins.

In some embodiments, the first and second component can each, independently, include the presence of a net charge on the component (and thus on the resultant liposome). This structural feature can provide liposome stability through electrostatic repulsion, which in turn can reduce the likelihood of aggregation or formation of multilayered liposomes, thus leading to liposomes of larger particle size. For example, anionic phospholipids can prevent aggregation of liposomes by electrostatic repulsion and provide enhanced shelf stability. However, upon I.V. administration, anionic lipids can be pulled out by the components of the blood. This in turn can lead to the breaking of the liposome and delivery of the previously encapsulated hydrophobic therapeutic agent to a blood compartment. Since blood compartments and the like are not recognized by the reticuloendothelial system (RES), then RES avoidance can be achieved. It is believed that the liposomes formed in the in vivo, fast breaking liposomal compositions and formulations described herein can enhance (e.g., increase) the degree and rate of transfer of the hydrophobic therapeutic agent to red blood cell (RBC), lipoproteins, HSA or WBC in blood relative to the degree and rate of transfer of the hydrophobic therapeutic agent into the RES.

In some embodiments, the first and second component can each, independently, include a fatty acid chain having a medium chain length, a fatty acid chain having one or more degrees of unsaturation, and a net charge.

In some embodiments, the presence of the first and second components can result in liposomal formulations that can behave in a manner similar to a DMSO or Cosolvent solution of the hydrophobic therapeutic agent.

Components such as the first and second components can include, without limitation, natural lecithins or phospholipids (e.g., phospholipids derived from any plant, animal, or bacterial source, e.g., derived from egg or soy sources and called egg or soy phosphatides, e.g., egg lecithin, egg phosphatidly ethanolamine, egg phosphatidly glycerol, egg phosphatidyl choline, soy phosphatidyl cholines, phosphatidic acid, plant monogalactosyl diglyceride (hydrogenated) or plant digalactosyl diglyceride (hydrogenated)); or synthetic lecithins (e.g., dihexanoyl-L-.alpha.-lecithin, dioctanoyl-L-.alpha.-lecithin, didecanoyl-L-.alpha.-lecithin, didodecanoyl-L-.alpha.-lecithin, ditetradecanoyl-L-.alpha.-lecithin, dihexadecanoyl-L-.alpha.-lecithin, dioctadecanoyl-L-.alpha.-lecithin, dioleoyl-L-.alpha.-lecithin, dilinoleoyl-L-.alpha.-lecithin, .alpha.-palmito, .beta.-oleoyl-L-.alpha.-lecithin, L-.alpha.-glycerophosphoryl choline). Other suitable phospholipids include dimyristoyl phosphatidyl choline (DMPC), phosphatidyl choline (PC), dipalmitoylphosphatidyl choline (DPPC), or distearoylphosphatidyl choline (DSPC); dimyristoylphosphatidylglycerol (DMPG) phosphatidyl ethanolamine, phosphatidylserine and phosphatidylinositol.

Examplary first and second components include Dimyristoyl Phosphatidyl Choline (DMPC), which is saturated 14 carbon chain; zwitterionic head group; Egg Phosphatidyl Choline (EPC, EggPC), which is a mixture of fatty acids; zwitterionic head group 16-18 carbon chain, about 42% saturated and about 57% unsaturated; Soy Phosphatidyl Choline (SPC), which is a mixture of fatty acids; zwitterionic head group 16-18 carbon chain about 17% saturated and about 81% unsaturated; or Egg Phosphatidyl Glycerol (EPG, EggPG), which includes essentially the same mixture as EPC, but a net negative charge on head group.

In certain embodiments, each of the first component and the second component can be, independently of one another, a natural lecithin or a phospholipid. For example, the first component can be egg phosphatidyl glycerol, and the second component can be soy phosphatidyl choline. As another example, the first component can be egg phosphatidly glycerol, and the second component can be DMPC.

In other embodiments, one or both of the first component and the second component can be a synthetic fatty acid chain having lipids, which are other than phospholipids. For example, synthetic fatty acid chains having a quaternary ammonium ion as the cationic portion and a sulfate group as the anionic portion.

In general, lyophilized liposomal compositions can include from about about 10 weight percent to about 90 weight percent (relative to the total weight of the composition) of the first component and the second component (e.g., from about about 10 weight percent to about 50 weight percent, from about about 20 weight percent to about 40 weight percent).

In some embodiments, the weight per cent ratio of the second component to the first component can be from about 1 to about 7 (e.g., from about 1 to about 5, from about 2 to about 5, from about 1 to about 3, from about 2 to about 3, from about 4 to about 5). The weight per cent ratio of the second component to the first component can be from about 2.2 to about 2.7. The weight per cent ratio of the second component to the first component can be from about 4.2 to about 4.8 ( e.g., 4.5).

In some embodiments, the number of moles of the first component can be less than the number of moles of the hydrophobic therapeutic agent. For example, the (ratio of the first component):hydrophobic therapeutic agent can be (from about 0.10 to about 0.95):1; e.g., (from about 0.50 to about 0.95):1; e.g., about (0.75):1.

In some embodiments, the number of moles of the first component can be about the same as the number of moles of the hydrophobic therapeutic agent.

In some embodiments, the number of moles of the first component can be from about 1.5 to about 6 times greater (e.g., from about 2 to about 5 times greater, from about 3 to about 4 times greater) than the number of moles of the hydrophobic therapeutic agent.

In some embodiments, the number of moles of the second component can be from about 2 to about 15 times greater (e.g., from about 2 to about 12 times greater, from about 2 to about 4 times greater, from about 4 to about 6 times greater, from about 6 to about 12 times greater, e.g., about 3 times greater, about 5 times greater, e.g., about 7 times greater, e.g., about 11 times greater) than the number of moles of the hydrophobic therapeutic agent.

In some embodiments, the number of moles of the first component can be less than the number of moles of the hydrophobic therapeutic agent (e.g., the (ratio of the first component):hydrophobic therapeutic agent can be (from about 0.10 to about 0.95):1; e.g., (from about 0.50 to about 0.95):1; e.g., about (0.75):1), and the number of moles of the second component can be from about 2 to about 15 times greater (e.g., from about 2 to about 12 times greater, from about 2 to about 4 times greater, from about 4 to about 6 times greater, from about 6 to about 12 times greater, e.g., about 3 times greater, about 5 times greater, e.g., about 7 times greater, e.g., about 11 times greater) than the number of moles of the hydrophobic therapeutic agent. For example, the number of moles of the first component can be less than the number of moles of the hydrophobic therapeutic agent (e.g., the (ratio of the first component):hydrophobic therapeutic agent can be (from about 0.50 to about 0.95):1, e.g., about (0.75):1), and the number of moles of the second component can be from about 2 to about 4 times greater (e.g., about 3 times greater).

In some embodiments, the number of moles of the first component can be about the same as the number of moles of the hydrophobic therapeutic agent, and the number of moles of the second component can be from about 2 to about 15 times greater (e.g., from about 2 to about 12 times greater, from about 2 to about 4 times greater, from about 4 to about 6 times greater, from about 6 to about 12 times greater, e.g., about 3 times greater, about 5 times greater, e.g., about 7 times greater, e.g., about 11 times greater) than the number of moles of the hydrophobic therapeutic agent. For example, the number of moles of the first component can be about the same as the number of moles of the hydrophobic therapeutic agent, and the number of moles of the second component can be from about 4 to about 6 times greater (e.g., about 5 times greater).

In some embodiments, the number of moles of the first component can be from about 1.5 to about 6 times greater (e.g., from about 2 to about 5 times greater, from about 3 to about 4 times greater) than the number of moles of the hydrophobic therapeutic agent, and the number of moles of the second component can be from about 2 to about 15 times greater (e.g., from about 2 to about 12 times greater, from about 2 to about 4 times greater, from about 4 to about 6 times greater, from about 6 to about 12 times greater, e.g., about 3 times greater, about 5 times greater, e.g., about 7 times greater, e.g., about 11 times greater) than the number of moles of the hydrophobic therapeutic agent. For example, the number of moles of the first component can be from about 2 to about 5 times greater (e.g., about 3 or about 4 times greater) than the number of moles of the hydrophobic therapeutic agent, and the number of moles of the second component can be from about 6 to about 12 times greater (e.g., about 7 or about 11 times greater).

In embodiments, the molar ratio of the hydrophobic therapeutic agent:first component:second component can be:

1:0.75:3
1:1:5
1:3:7
1:4:11

In some embodiments, the weight per cent ratio of the first component and the second component to the hydrophobic therapeutic agent can be from about 2 to about 50 (e.g., from about 10 to about 50, from about 15 to about 25).

Cryoprotectants

Cryoprotectants provide protection against freezing of the aqueous formulations (e.g., during storage). Suitable cryoprotectants include glycine, glycerol; sugars (e.g., monosaccharides, disaccharides, or polysaccharides, e.g., glucose, fructose, lactose, trehalose, maltose, maltotriose, palatinose, lactulose or sucrose); or polyhydroxy alcohols (e.g., mannitol, sorbitol). In general, lyophilized liposomal compositions can include from about 50 weight percent to about 75 weight percent (relative to the total weight of the composition) of a cryoprotectant. In some embodiments, the weight per cent ratio of the cryoprotectant to the first component and the second component can be from about 1.5 to about 5 (e.g., from about 2 to about 3).

In some embodiments, the cryoprotectant is a monosaccharide, disaccharide or polysaccharide (e.g., glucose, fructose, lactose or trehalose). In certain embodiments, the presence of a monosaccharide, disaccharide or polysaccharide in the liposomal formulations can yield liposomes having relatively small and narrow particle size distribution (e.g., from about 130 nm to less than about 200 nm), in which the hydrophobic therapeutic agents can be stably encapsulated into the liposome in a relatively efficient manner (e.g., with an encapsulation efficiency of greater than or equal to about 80 per cent, e.g., greater than or equal to about 90 per cent, e.g, greater than or equal to about 95 per cent).

In general, encapsulation efficiency can be estimated as follows: (1) a liposomal solution is prepared, e.g., using the methods described herein, containing a known amount of a hydrophobic therapeutic agent; (2) the concentration of the hydrophobic therapeutic agent in the liposomal solution is measured; (3) the resultant liposomal solution is filtered through a 0.22μ filter, after which liposomes of approximately nanometer particle size distribution are retained in the filtered lipsomal solution; (4) the concentration of the hydrophobic therapeutic agent in the filtered liposomal solution is measured; and (5) the encapsulation efficiency is determined by dividing the hydrophobic therapeutic agent concentration obtained in step (4) by the hydrophobic therapeutic agent concentration obtained in step (2).

Anti-Oxidants, Additional Ingredients, and Exemplary Lyophilized Compositions

In some embodiments, each of the first anti-oxidant and the second anti-oxidant can be, independently of one another, butylated hydroxytoluene (BHT), butylated hydroxyl anisole (BHA), α-tocopherol or acyl esters thereof, pegylated vitamin E (e.g., TPGS), or ascorbyl palmitate. In preferred embodiments, the anti-oxidant is a hydrophobic anti-oxidant (e.g., BHT, BHA, α-tocopherol, or ascorbyl palmitate). In other embodiments, the lyophilized liposomal compositions can include more than two anti-oxidants (3, 4, 5, 6, 7, 8, 9, or 10 anti-oxidants).

In some embodiments, lyophilized liposomal compositions can further include one or more surfactants (e.g., pegylated (PEG) vitamin E of various chain lengths, tyloxopol and pegylated (PEG) derivatives thereof, or monosaccharides having aliphatic chains of 5-15 carbons).

Exemplary lyophilized liposomal compositions include those delineated in Table 1.

TABLE 1

| Ingredient | Amount % (w/w) |
|---|---|
| Hydrophobic therapeutic agent | about 0.500 to about 2.500 |
| First Component | about 5 to about 15 |
| Second Component | about 15 to about 25 |
| First Anti-oxidant | about 0.005 to about 0.020 |
| Second Anti-oxidant | about 0.025 to about 0.050 |
| Cryoprotectant | about 50 to about 75 |

Substantially Homogeneous Liposomal Solutions

In some embodiments, substantially homogeneous liposomal solutions or substantially homogeneous liposomal formulations can include one or more hydrophobic therapeutic agents, a first component, a second component, a cryoprotectant, a first anti-oxidant, a second anti-oxidant, and water. Particular hydrophobic therapeutic agents, first components, second components, cryoprotectants, first anti-oxidants, and second anti-oxidants can be selected as described elsewhere.

In some embodiments, the substantially homogeneous liposomal formulations can include at least about 70 weight/volume per cent of water (e.g., at least about 75 weight/volume per cent, at least about 80 weight/volume per cent, at least about 85 weight/volume per cent, at least about 90 weight/volume per cent, at least about 95 weight/volume per cent).

In general, the concentration of the hydrophobic therapeutic agents in the substantially homogeneous liposomal formulations can depend, e.g., upon the nature of the hydrophobic therapeutic agent. In some embodiments, the formulations can include from about 0.050 weight/volume (w/v) per cent to about 0.500% weight/volume (w/v) per cent of the hydrophobic therapeutic agent, thereby providing a liposomal solution having from about 0.5 mg/ml to about 10.0 mg/ml (e.g., from about 0.5 mg/ml to about 8.0 mg/ml, e.g., 2 mg/ml) of the hydrophobic therapeutic agent.

In all embodiments, the formulations are capable of being diluted indefinitely with water without precipitation of the hydrophobic therapeutic agent.

In general, liposomal solutions containing liposomes of nanometer average particle size distribution form water-clear, translucent solutions (e.g., substantially homogeneous liposomal solutions or substantially homogeneous liposomal formulations). Precipitation of the hydrophobic therapeutic agent can therefore be monitored using qualitative techniques, e.g., visually monitoring a shaking liposomal solution for the formation of, e.g., a cloudy or milky dispersion. Precipitation of the hydrophobic therapeutic agent can also be monitored using the quantitative techniques (also in conjunction with qualitative techniques) described herein. For, example, a substantial change in the concentration of a filtered and unfiltered liposomal solution can indicate precipitation of the hydrophobic therapeutic agent.

Exemplary formulations include those delineated in Table 2.

TABLE 2

| Ingredient | Amount % (w/v) |
|---|---|
| Hydrophobic therapeutic agent | about 0.050 to about 0.500 |
| First Component | about 0.5 to about 5.0 |
| Second Component | about 1.5 to about 6.0 |
| First Anti-oxidant | about 0.001 to about 0.01 (e.g., |
| | about 0.001 to about 0.005) |
| Second Anti-oxidant | about 0.001 to about 0.01 (e.g., |
| | about 0.004 to about 0.008) |
| Cryoprotectant | about 2 to about 15 (e.g., about 5 to |
| | about 15) |
| Water | about 70 to about 90 |

In some embodiments, some or all of the first components, second components, cryoprotectants, first anti-oxidants, and second anti-oxidants as well as other additives present in the lyophilized liposomal compositions, the substantially homogeneous liposomal solutions or the substantially homogeneous liposomal formulations can also be selected from those described in U.S. Pat. No. 4,816,247 and U.S. Pat. No. 6,890,555, and U.S. Pat. No. 7,135,193B2 all of which are incorporated by reference herein.

In all embodiments, the liposomes have an average particle size distribution of less than about 5,000 nm, so as to minimize the likelihood of obstructing lung capillaries. In general, the liposomes have an average particle size distribution of from about 30 nm to about 500 nm (e.g., from about 50 nm to about 300 nm, e.g., about 200 nm, about 30 nm to at most about 200 nm, less than about 200 nm).

In general, conventional liposomal formulations are preferentially taken up by the reticuloendothelial system (RES) organs such as the liver and spleen. In some instances, only 10 to 20% of the drug is available in the systemic circulation. If the particle size increases, as a consequence of normal aging of conventional formulations, the likelihood of RES uptake is further increased.

When RES uptake of the liposomes occurs, a substantial portion of the encapsulated hydrophobic therapeutic agent is not available to the target tissue since it is localized in the RES. In embodiments, the new liposomal formulations can be “fast breaking” in that the hydrophobic therapeutic agent-liposome combination is stable in vitro but when administered in vivo, the hydrophobic therapeutic agent is rapidly released into the bloodstream where it associates with serum lipoproteins, red blood cells, and human serum albumin. In some embodiments, the liposomal formulations (liposomes) can be fast breaking and rapidly release the hydrophobic therapeutic agent into the bloodstream to associate with, e.g., red blood cell (RBC), lipoproteins, HSA or WBC in blood upon in vivo administration. While not wishing to be bound by theory, it is believed that this rapid release can prevent the hydrophobic therapeutic agent from being accumulated in non-target tissues such as the liver, spleen, or bone marrow where liposomes otherwise have a tendency to concentrate. The “fast breaking” nature of the preferred liposomes may also be associated with the manner in which the hydrophobic therapeutic agent interacts with the lipid bilayer of the liposomes that are formed in the formulations described herein.

The compositions and formulations described herein can include the hydrophobic therapeutic agents themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

In some embodiments, lyophilized liposomal compositions can be prepared by process, which includes:

(i) combining a hydrophobic therapeutic agent, a first component, and a second component in an organic solvent to form a first combination;

(ii) combining the first combination with a water phase to form a second combination (e.g., a liposomal solution);

(iii) removing the organic solvent from the second combination to form a third combination; and

(iv) lyophilizing (e.g., freeze-drying) the third combination.

Preferred organic solvents include those that can be removed with relative ease and practicality by evaporation under reduced pressure (e.g., aspirator pressure or low vacuum, e.g., from about 1 mmHg to about 50 mmHg). Exemplary organic solvents include, for example, C₁₋₆ straight chain and branched alcohols (e.g., ethanol or isopropanol or t-butanol); C₁₋₆ straight chain and branched halo alkanes (e.g., chlorinated alkanes, e.g., chloroform or methylene chloride); C₁₋₆ straight chain and branched alkyl esters (e.g., ethyl acetate).

In some embodiments, the first combination can include one or more antioxidants (e.g., two anti-oxidants).

In some embodiments, the water phase can include a cryoprotectant (e.g., lactose).

In some embodiments, the second combination can be a liposomal solution, and the process can further include the step of reducing the particle size distribution of the liposomes (e.g., reducing the average particle size distribution of the liposomes to about 50 nm to about 500 nm (e.g., from about 50 nm to about 300 nm, e.g., (to at most about 200 nm, less than 200 nm) to form, e.g., a “substantially homogeneous liposomal solution of the hydrophobic therapeutic agent” or a “substantially homogeneous liposomal formulation of the hydrophobic therapeutic agent”).

In some embodiments, step (iii) can include removing some or substantially all of the organic solvent, e.g., by distillation; evaporation under reduced pressure (e.g., aspirator pressure or low vacuum, e.g., from about 1 mmHg to about 50 mmHg); or tangential flow filtration.

In certain embodiments, step (iii) can include performing a tangential flow filtration (TFF). In preferred embodiments, the organic solvent is preferably a water miscible organic solvent (e.g., ethanol, iso-propanol, n-propanol, propylene glycols, polyethylene glycols). Typically, the organic solvent can be removed after about 5 to 10 (e.g., 5-6) passes of the filter membrane through a liposomal solution.

The use of TFF in the processes described herein can have one or more of the following advantages. For example, TFF is generally scalable and is flexible with respect to the organic solvent that is to be removed. TFF can typically be used to remove essentially any low molecular weight, water-miscible organic solvent from liposomal solutions having a total volume of from about 100 mL to about 10,000 liters (L) (e.g., from about 100 mL to about 1,000 mL (e.g., 1-3, 30-50 liters). In addition, TFF process itself does not involve a solvent evaporation step. Therefore, the use of TFF can potentially reduce the operating costs associated with conducting the processes described herein (e.g., tangential flow filtration can be, but need not be conducted within the confines of specially designed and costly facilities (e.g., explosion-proof facilities), which are typically needed (and sometimes required) for accommodating solvent removal equipment such as vacuum systems, heating mantles, condensation towers. As a further example, the use of tangential flow filtration allows the processes described herein to be conducted essentially as a one-pot operation, thereby minimizing the likelihood for bulk transfer of liposomal solutions at intermediate stages of the process.

In some embodiments, the process can further include the step of aseptic filtration.

In some embodiments, the process can provide liposomes having sufficiently small and narrow average particle size distribution (e.g., less than about 200 nanometers (nm)) such that the liposomal formulations can be manufactured without filtering to separate off larger particles or utilizing other mechanical methods of obtaining a narrow distribution of particle size distribution.

In other embodiments, lyophilized liposomal compositions can be prepared by process, which includes:

(i) combining a hydrophobic therapeutic agent, a first component, and a second component in an organic solvent to form a first combination;

(ii) removing the organic solvent from the first combination to form a second combination (e.g., removing some or substantially all of the organic solvent to form, e.g., a thin film);

(iii) combining the second combination with a water phase to form a third combination; and

(iv) lyophilizing the third combination, thereby preparing the lyophilized liposomal composition.

In general, the lyophilized compositions can be stored at from about 2° C. to about 37° C. (e.g., about 2° C. to about 8° C.) for about 2 years or more. The lyophilized compositions can also be stored at lower temperatures, e.g., at about −20° C. to −70° C.

In addition, the processes described herein can be used to prepare emulsions, vescicles, or high molecular weight assemblies that have one or more hydrophobic therapeutic agents.

In general, substantially homogeneous liposomal aqueous formulations or solutions can be prepared by reconstituting the corresponding lyophilized liposomal compositions described herein with an aqueous vehicle. The reconstituted compositions can be diluted indefinitely with water and are typically stable physically and chemically at room temperature for a period of about one week.

The liposomal aqueous formulations are typically administered parenterally. Injection can be intravenous, subcutaneous, intramuscular, intrathecal, or even intraperitoneal. The liposomal formulations can be applied by transdermal, sublingual, oral, occular, vaginal and the colonic routes. In certain embodiments, the liposomal aqueous formulations can be administered by aerosol intranasaly, intrabronchially, intraalvelorly, or intrapulmonarily. The compositions can be packed in vials for reconstitution with sterile water prior to injection may also contain minor amounts of nontoxic, auxiliary substances such as pH buffering agents, preservatives, chelating agents, antioxidants, osmotic pressure adjusting agents and the like.

Dosages can range from about 0.01 mg/Kg to about 1000 mg/Kg, (e.g., from about 0.01 mg/kg to about 100 mg/kg, from about 0.01 mg/kg to about 10 mg/Kg, from about 0.05 mg/kg to about 10 mg/Kg, from about 0.1 mg/kg to about 10 mg/kg) every 0.5 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, New York, 537 (1970). The methods herein contemplate administration of an effective amount of hydrophobic therapeutic agent to achieve the desired or stated effect. Typically, the formulations described herein will be administered from 1 to 6 times per day or alternatively, as a continuous infusion. In certain embodiments, liposomal aqueous formulations can be administered as a bolus (e.g., administered over the course of about 1 minute) or a slow bolus (e.g., administered over the course of about from about 15 minutes to about 20 minutes). Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of the formulations described herein may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions and formulations of this invention may contain any conventional non-toxic pharmaceutically-acceptable carriers, diluents or adjuvants (e.g., the compositions and formulations can be prepared as, stored as, or administered in the form of a pharmaceutical composition). In some cases, the pH of the formulation may be adjusted with pharmaceutically acceptable acids, bases or buffers to enhance the stability of the formulated compound or its delivery form.

In some embodiments, the formulations described herein can be coadministered with one or more other therapeutic agents. In certain embodiments, the additional agents may be administered separately, as part of a multiple dose regimen, from the compounds of this invention (e.g., sequentially, e.g., on different overlapping schedules with the administration of the formulations described herein). Alternatively, these agents may be part of a single dosage form, mixed together with the hydrophobic therapeutic agents in a single composition. In still another embodiment, these agents can be given as a separate dose that is administered at about the same time that the formulations described herein are administered (e.g., simultaneously with the administration of the formulations described herein).

The invention will be further described in the following examples. It should be understood that these examples are for illustrative purposes only and are not to be construed as limiting this invention in any manner.

### EXAMPLES

### Candidate Hydrophobic Therapeutic Agents

Compound 1: (5-fluoro-2-methyl-N-[4-(5H-pyrrolo[2,1-c][1,4]benzodiazepin-10(11H)-yl carbonyl)-3-chlorophenyl]benzamide; Molecular Formula—C₂₇H₂₁ClFN₃O₂; Molecular Weight—473.9; melting point is 180° C.-184° C.

Compound 1 is a nonpeptide vasopressin receptor antagonist and is insoluble in aqueous solutions throughout the physiological pH range (1.2 to 7.5). It has limited solubility in partially aqueous solvents of pharmaceutically acceptable hydrophilic and lipophilic solvents. The solubilities of Compound 1 at 25° C. in various pharmaceutical systems is provided in Table 1.

TABLE 3
Solubility of Compound 1 in Pharmaceutical Solvents at 25° C.

| Excipients | Solubility (mg/ml) |
|---|---|
| SGF without enzyme | BQL+ |
| SIF without enzyme | BQL+ |
| 10 mM Phosphate buffer (pH 7.4) | BQL+ |
| Aqueous solution of Poloxamer 188 | BQL+ |
| (10, 20 and 30%) | |
| Benzyl Alcohol | 21.03 |
| Benzyl Benzoate | 3.41 |
| Ethanol | 1.89 |
| Triacetin | 2.70 |
| Cremophor EL | 5.10 |
| Safflower oil | BQL+ |
| Soybean oil | BQL+ |
| Olive oil | 0.03 |
| Oleic acid | 0.04 |
| Ethyl oleate | 0.20 |
| Neobee M5** | 0.52 |
| Labrasol*** | 7.50 |
| Miglyol 812** | 0.29 |
| Gelucire 48109**** | 2.26 |
| PEG 400 | 26.30 |
| PEG 300 | 14.86 |
| PEG 300/Alcohol (50:50) | 8.75 |
| PEG 300/Alcohol/Water (40:30:30) | 0.28 |
| Propylyne glycol | 0.60 |
| Propylene glycol Laurate | 0.68 |
| PEG 400/Benzyl Alcohol (80:20) | 14.60 |
| Sodium Lauryl sulfate | 0.02 |
| Human Serum Albumin | 0.016 |

BQL+ = Below quantitation limit Quantification limit = (1 ng/ml)
**Medium chain (C5-C8) triglycerids with different mixtures of medium chains
***Saturated polyglycolized C8-C10 glycerides
****Saturated polyglycolized glycerides obtained from hydrogenated vegetable oils and consisting of glycerides and polyethylene glycol esters

Table 3 shows that the solubilities (mg/ml) of Compound 1 in PEG 400, PEG 300, Benzyl Alcohol, Benzyl Benzoate, Triacetin and Ethanol are 26.3, 14.86, 21.03, 3.41, 2.70 and 1.89 respectively, whereas a 50:50 combination of PEG 300 and Ethanol lowered the solubility to 8.75 mg/ml. An addition of water to obtain a PEG 300/Ethanol/Water (40:30:30) composition lowers the solubility to 0.28 mg/ml.

In additional combinations as cosolvent systems, of PEG 400 or 300, Benzyl Alcohol, Ethanol and low amounts of bile salts (e.g. Sodium Deoxytauro Cholate), Compound 1 precipitated upon an addition of water at 30% levels in the final aqueous-organic mixture.

Compound 2: But-2-ynoic acid[4-(3-bromo-phenylamino)-quinazolin-6-yl]-amide mesylate; Formula: C₁₈H₁₃BrN₄O.CH₄O₃S

### Example 1

### General Tangential Flow Filtration Procedure for Preparing Lyophilized Liposomal Compositions

General Procedure: The hydrophobic therapeutic agent, phospholipids, and antioxidants are dissolved in dehydrated alcohol to form a hydrophobic therapeutic agent-phospholipid complex. When the alcoholic solution is diluted by an aqueous lactose solution, a liposomal solution with alcohol is formed. The alcohol is removed by repeated molecular sieving operations through tangential flow filtration equipment (TFF). The resultant aqueous liposomal solution is passed through a high-pressure homogenizer to resuce the particle size distribution to the submicron range and is filtered aseptically through a 0.22 μm filter and filled in the vials. The vial contents are lyophilized for chemical stability.

The scale-up batches using the TFF method were manufactured at 50 liter scale of the bulk liposomal solution prior to lyophilization. Representative data for Compound 1 liposomal lyophilized formulation with molar ratio composition of Drug:EggPG:DMPC (1:4:11) are provided. Compositions and formulations having other molar ratio compositions than those described here can be manufactured by the TFF process.

Tangential Flow Filtration Process Summary for Lyophilized Liposomal Compound 1 (15 mg/vial) (Representative Example)

1). Prepare Alcoholic Solution, 4 mg/ml Compound 1 and Phospholipids.

2). Add water for injection (WFI) slowly to 1) at a volume of 3 times the Alcoholic Solution of 1) and mix to provide Aqueous-Alcoholic Liposomal Solution 1.0 mg/ml Compound 1.

3). Perform Tangential Flow Filtration (TFF) on Aqueous-Alcoholic Liposomal Solution of 2) with at least 6-8 exchanges with WFI (using in-process test for alcohol) to provide Aqueous Liposomal Solution 1.0 mg/ml Compound 1.

4). Concentrate Aqueous Liposomal Solution of 3) by TFF to 45% of the initial volume to provide Aqueous Liposomal Solution 2.2 mg/ml Compound 1.

5) Add solid Lactose & WFI to Aqueous Liposomal Solution 2.2 mg/ml Compound 1 of 4), adjust the potency to 1.8 mg/ml Compound 1 and perform high pressure homogenization to provide Aqueous Liposomal Solution with Lactose, 1.8 mg/ml Compound 1.

6. Filter Aqueous Liposomal Solution with Lactose, 1.8 mg/ml Compound 1 of 5) through 0.45μ and 0.22μ filters to provide Filtered Liposomal Solution with Lactose, 1.8 mg/ml Compound 1.

7. Perform in process HPLC potency determination on Filtered Liposomal Solution with Lactose, 1.8 mg/ml Compound 1 of 6) and adjust potency to 1.58 mg/ml Compound 1 by adding 25% Lactose Solution & WFI. Refilter through 0.22μ into a sterile area to provide Sterile Liposomal Solution with Lactose, 1.58 mg/ml Compound 1.

8. Fill the Sterile Liposomal Solution with Lactose, 1.58 mg/ml Compound 1 of 7) at 10.5 ml/vial. Lyophilize to provide Lyophilized Liposomal Compound 1 Vials.

Table 4 shows Pilot Batch for Compound 1 Liposomal Solution (TFF Operation Results) (Ethanol Removal Efficiency as a Function of TFF Passes)

TABLE 4

| | | | Inlet | Outlet | Permeate | % Alcohol |
|---|---|---|---|---|---|---|
| Permeate Sample | Product Temp | Flux | Pressure | Pressure | Pump | in |
| No.+ | ° C. | ml/min | psi | psi | Speed % | Permeate |
| H₂O recirculation | 47 | 2300 | 16 | 5 | recirculate | — |
| initial 0 | 45 | 510 | 25 | 18 | 24 | — |
| 1 | 34 | 720 | 27 | 9 | 28 | 17.65 |
| 2 | 34 | 800 | 26 | 8 | 30 | — |
| 3 | 34 | 800 | 26 | 8 | 30 | — |
| 4 | 32 | 840 | 25 | 8 | 30 | — |
| 5 | 32 | 820 | 27 | 8 | 30 | — |
| 6 | 33 | 810 | 28 | 8 | 31 | 0.042 |
| 7 | 33 | 700 | 29 | 8 | 31 | — |
| 8 | 33 | 600 | 26 | 6 | 21 | 0.038 |

Concentration of Retentate

| | | | | | | |
|---|---|---|---|---|---|---|
| 16 L to 10 L | 30 | 360 | 25 | 3 | 24 | — |
| 10 L to 8.6 L | 30 | 340 | 25 | 3 | 27 | — |

+Permeate samples are collected at the end of 12-13 liter exit of the permeate.

### Summary of Process Parameters of Lyophilized Liposomal Compound 1 Vials by TFF Method

| Initial Alcoholic-H₂0 Liposomal | 16,000 | ml |
|---|---|---|
| Solution Volume = | | |
| Final Volume of Reduced Alcohol | | |
| Liposomal Concentrate Solution = | 8,600 | ml |
| Final Adjusted Volume (after solid | | |
| lactose +WFI Addition = | 10,000 | ml |
| Total Time for TFF Operation = | 3 | hrs |
| Appearance | | |
| Initial Alcoholic-H₂0 Liposomal = | milk like | |
| Solution Final Liposomal Solution (TFF + | | |
| Concentration + Lactose + H₂0) = | clear translucent | |
| Filterability | | |
| Prior to Microfluidization = | 40 | ml thro' 0.45 μ and |
| | 20 | ml thro' 0.45/0.22 μ |
| Post Microfluidization = | >>40 | ml thro' 0.45 μ and |
| (2 passes at 18,500 psi) | 30 | ml thro' 0.45/0.22 μ |
| Potency Value of Final Liposomal | | |
| Bulk Solution | | |
| Unfiltered Solution = | 1.523 | mg/ml |
| Filtered Through 0.22 μ Millipore 200 = | 1.513 | mg/ml |
| Vial Fill Volume for Lyophilization = | 10.5 ml | |

| | |
|---|---|
| Manufacturing Efficiency = | 93% (based on total VPA-985 |
| | used in start and total obtained |
| | in vials) |
| Freeze Drying | |

Break the Vacuum by N₂, Stopper, Seal and Store the vials at 2-8° C.
Evaluation of Freeze Dried Vials

| | |
|---|---|
| Appearance of Cake | white cake with slight yellow color |
| Moisture - 3.99% | |
| Alcohol - 0.1% | |
| Constitutability | constitutes to clear solution in 15 seconds |
| | the solution was free of any precipitates at |
| | least for 4 days observation period |
| Constituted | pH = 5.54 and 297 mosm osmolarity |
| Solution | particle size distribution (By Nicomp) |

| | |
|---|---|
| Bimodel | Distribution |
| 89 nm | 69 vol % |
| 11 nm | 3l vol % |

| | | |
|---|---|---|
| Compound 1 Potency on Vial basis = | 15.91 | mg/vial |
| Total No. Vials Manufactured = | 950 | vials |

Initial Alcoholic-H₂0 Liposomal Solution Volume=16,000 ml
Final Volume of Reduced Alcohol

Liposomal Concentrate Solution=8,600 ml
Final Adjusted Volume (after solid lactose +WFI Addition=10,000 ml
Total Time for TFF Operation=3 hrs
Appearance
   Initial Alcoholic-H₂0 Liposomal Solution=milk like
   Final Liposomal Solution (TFF+Concentration+Lactose+H₂0)=clear translucent
Filterability
   Prior to Microfluidization=40 ml thro' 0.45μ and 20 ml thro' 0.45/0.22μ
   Post Microfluidization=>>40 ml thro' 0.45μ and (2 passes at 18,500 psi) 30 ml thro' 0.45/0.22μ
Potency Value of Final Liposomal Bulk Solution
   Unfiltered Solution=1.523 mg/ml
   Filtered Through 0.22μ Millipore 200=1.513 mg/ml
Vial Fill Volume for Lyophilization=10.5 ml
Manufacturing Efficiency=93% (based on total VPA-985 used in start and total obtained in vials)
Freeze Drying
Break the Vacuum by N₂, Stopper, Seal and Store the vials at 2-8° C.
Evaluation of Freeze Dried Vials
   Appearance of Cake: white cake with slight yellow color
   Moisture-3.99%
   Alcohol-0.1%
   Constitutability: constitutes to clear solution in 15 seconds the solution was free of any precipitates at least for 4 days observation period
   Constituted: −pH=5.54 and 297 mosm osmolarity Solution -particle size distribution (By Nicomp) Bimodel Distribution 89 nm 69 vol % 11 nm 31 vol %
Compound 1 Potency on Vial basis=15.91 mg/vial
Total No. Vials Manufactured=950 vials

Representative compositions prepared by TFF: Tables 5 and 6 show representative lyophilized liposomal compositions containing Compound 1 and Compound 2, respectively, that were prepared by TFF method.

TABLE 5

| Component | Function | w/v %* |
|---|---|---|
| Molar Ratio | | 1:4:11 |
| Compound 1:EPG:SPC | | |
| Compound 1 | Hydrophobic therapeutic | 0.2 |
| | agent | |
| Egg Phosphatidyl glycerol (EPG) | First Component | 1.232 |
| Dimyristoyl Phosphatidyl | Second Component | 2.984 |
| choline (DMPC) | | |
| BHT | Antioxidant | 0.002 |
| Ascorbyl palmitate | Antioxidant | 0.005 |
| Lactose | Cryoprotectant | 9.0 |
| Water for Injection | Diluent for Reconstitution | >86 |
| Total grams of lipids/200 mg | | 4.216 |
| Compound 1 | | |

*Composition of the liposomal solution upon reconstitution by adding water

TABLE 6

| Component | Function | w/v %* |
|---|---|---|
| Molar Ratio | | 1:2:5.5 |
| Compound 2:EPG:SPC | | |
| Compound 2 | Hydrophobic therapeutic | 0.4 |
| | agent | |
| Soy Phosphatidyl glycerol (SPG) | First Component | 1.232 |
| Soy Phosphatidyl choline (SPC) | Second Component | 2.984 |
| BHT | Antioxidant | 0.002 |
| Ascorbyl palmitate | Antioxidant | 0.002 |
| Lactose | Cryoprotectant | 9.0 |
| Water for Injection | Diluent for Reconstitution | >86 |
| Total grams of lipids/200 mg | | 2.1 |
| Compound 2 | | |

*Composition of the liposomal solution upon reconstitution by adding water

### Example 2

### General Procedure for Preparing Liposomal Formulations (Thin Film Technology by Rotovap)

The IV formulation is presented as a vial containing sterile lyophilized liposomal powder equivalent to 20 mg of Compound 1 per vial. Based on water insolubility and other physicochemical properties of Compound 1, a liposomal formulation for an IV administration of the drug was selected. By an addition of 10 mL of Water for Injection, a water-like liposomal solution (with about 200 nm average size liposomes) containing 2 mg/ml of Compound 1 is obtained. The compositions of Prototype A (1:3:7 molar ratio) and Prototype B (1:4:11 molar ratio) are provided in Table 7. They were manufactured by Thin Film Rotovap method as described below. The stability data of a representative batch is provided in Table 8. The reconstitutability parameters for the liposomal particle size distribution are also provided.

Representative Lyophilized Liposomal Compositions of Hydrophobic Therapeutic Agent (Thin Film Rotovap Procedure):

TABLE 7

| | | Prototype A | Prototype B |
|---|---|---|---|
| Component | Function | w/v %* | w/v %* |
| Molar Ratio | | 1:3:7 | 1:4:11 |
| Compound 1:EPG:SPC | | | |
| Compound 1 | Hydrophobic | 0.2 | 0.2 |
| | therapeutic | | |
| | agent | | |
| Egg Phosphatidyl | First Component | 0.975 | 1.232 |
| glycerol (EPG) | | | |
| Soy Phosphatidyl choline | Second | 2.363 | 2.984 |
| (SPC) | Component | | |
| BHT | Antioxidant | 0.002 | 0.002 |
| Ascorbyl palmitate | Antioxidant | 0.005 | 0.005 |
| Lactose | Cryoprotectant | 9.0 | 9.0 |
| Water for Injection | Diluent for | >87 | >86 |
| Total grams of lipids/ | Reconstitution | 3.338 | 4.216 |
| 200 mg Compound 1 | | | |

*Composition of the liposomal solution upon reconstitution by adding water

Liposome Manufacture (Thin Film Technology by Rotovap): Representative Steps:

1. Dissolution: Hydrophobic Therapeutic Agent (HTA)—Phospholipids in Organic Solvent (Process Controls: ˜Low Dissolved O₂ Levels; ˜Mixing Rate and Temperature; ˜Solvent Exposure).

2. Solvent Removal and Deposition of HTA-Lipid Complex as Thin Film (Process Controls: ˜Evaporation Rate; ˜Establish Residual Solvent Levels; ˜Film Thickness and Porosity).

3. Controlled Hydration and Coarse Liposome Formation (Process Controls: ˜Rate of Film Wetting; ˜Hydrating Solvent Temperature; ˜Mixing).

4. Particle Size Reduction (Process Controls: ˜Chamber Pressure; ˜Inlet, Chamber and Outlet Temp.; ˜Number of Passes).

5. Prefiltration (Coarse Filter) and Non-Dispersed Drug Removal (Process Controls: ˜Filtration Pressure; ˜Rate and Temperature).

6. Aseptic Filtration (Process Controls: ˜Filterability Test; ˜Filtration Pressure and Rate; ˜Potency Analysis).

7. Vial Filling (Process Controls: ˜Bulk Solution Temp and Homogeniety; ˜Weight Control).

8. Lyophilization (Process Controls: Customize Primary, Secondary; Tertiary Drying Steps; ˜Moisture Analysis).

9. Physical and Chemical Tests for Release (Process Controls: ˜Monograph Tests; ˜Particle Size Reproducibility).

Formal Stability Data for Compound 1 for Injection# (15 mg/vial) Lyophilized Formulation (1:4:11 Molar Ratio):

TABLE 8

| | Liposomal* 15 mg/vial | | | |
|---|---|---|---|---|
| | | % | | |
| Storage Condition | Potency (mg/vial) | remain | TRC (Area %) | pH |
| Initial | 15.46 | 103.7 | 1.44 | 6.10 |
| 5 C. 3M | 15.04 | 100.3 | 1.84 | 6.19 |
| 25 C./60% RH 1 M | 15.32 | 102.15 | 1.75 | 6.12 |
| 25 C./60% RH 3 M | 14.92 | 99.47 | 2.82 | 6.20 |
| 25 C./60% RH 6 M | 14.71 | 98.03 | 3.44 | 6.07 |
| 25 C./60% RH 9 M | 14.35 | 95.68 | 3.83 | 6.20 |
| 40 C./75% RH 1 M | 14.86 | 99.07 | 2.91 | 6.02 |
| 40 C./75% RH 3 M | 14.09 | 93.96 | 6.16 | 5.98 |
| LCAB 1 M | 12.09 | 80.6 | 19.37 | 5.52 |

# Molar Ratio + Drug:EPG:DMPC 1:4:11
*Liposomal stability data is for the batch where:
Film stored at 2-8 C. for 1 M
Lyophilized batch stored at room temp. and placed on formal stability after 1M
Thus Actual age of the sample is formal storage point plus two months

Reconsitution of Liposomal Solution and Particle Size Parameters:

The lyophilized liposomal cake reconstituted to a clear to translucent solution, within 30 seconds of an addition of WFI and mixing. If there was any foam it subsided within 10 minutes. The pH of the solution was between 6.0 and 6.2. The particle size distribution was measured by Nicomp Submicron Particle Size Analyzer using the dynamic light scattering method. For the formulation the representative Nicomp plot indicates monomodal size distribution of fine liposomes.

The plot showed:
Mean Diameter: 39 nm
Std. Deviation: 19 nm
Coeff. Of Variation: 0.499
Chi Square Value: 23
Cumulative Results:
75% of Particles <46 nm
99% of Particles <105 nm

### Example 4

### Studies to Define Molar Compositions of Phospholipid Mixtures

Initial experiments indicated that a combination of EPG (anionic phospholipid) and DMPC (semi-synthetic saturated hydrocarbon chain zwitterionic phospholipid) provided means of solubilizing Compound 1. Further experiments were conducted to define an optimum molar ratio of Drug:EPG:DMPC which provides acceptable drug loading, chemical and physical stability and manufacturability. The examples of other anionic phospholipids and zwitterionic phospholipids combinations for stable manufacturable liposomes encapsulating 1.5 to 2.0 mg/ml are provided in table 9.

TABLE 9
Lipid Ratios for Compound 1 Lyophilized Liposomal Formulations*
(Reconstituted Liposomal Solution of Compound 1 at 1.5 to 2.0 mg/ml)

| | | Formulation Prototypes Molar Ratio | | | | | | | | |
|---|---|---|---|---|---|---|---|---|---|---|
| Component | Function | A | B | C | D | E | F | G | H | I |
| Cmpd 1 | Hydrophobic Drug | 1 | 1 | 1 | 1 | 1 | 1 | 1 | 1 | 1 |
| EggPG | Anionic Complexant | 3 | 3 | 4 | — | 3 | — | — | — | — |
| SoyPG | Anionic Complexant | — | — | — | 3 | — | — | 3 | — | — |
| DMPG | Anionic Complexant | — | — | — | — | — | 3 | — | 1.5 | 2.0 |
| DPPG | Anionic Complexant | — | — | — | — | — | — | — | 1.5 | 1.0 |
| DMPC | Zwitterionic Complexant | 7 | 11 | 11 | 11 | — | 7 | 11 | 11 | — |
| DPPC | Zwitterionic Complexant | — | — | — | — | — | — | — | — | 10 |
| Soy PC | Zwitterionic Complexant | — | — | — | — | 7 | — | — | — | — |

DMPG = Dimyristoyl Phosphatidyl Glycerol
DPPC = Dipalmitoyl Phosphatidyl Choline
*Additionally, they contain lactose or other disaccharides as cryoprotectants and BHT and ascrobyl palmitate or other compounds as antioxidants.

### Example 5

### Biological Evidence For The Reticuloendothelial (Res) Avoiding Design Of Liposomes

When typical, conventional drug carrier liposomes are administered by the IV route, in general about 80-90% of the drug is sequestered by the reticuloendothelial organs (i.e. liver, spleen, bone marrow etc.) and very small amount of the drug is available in the systemic circulation. This can defeat the purpose of the therapy, if the target is other than RES.

For Compound 1 liposomal design the phospholipid molecular type and composition were selected such that liposomes are stable in vitro or on shelf, and break up and deliver the drug to one or more components of the blood which become the circulatory drug reservoir and as a result the dosage form behaves like a simple solution providing majority of the dose to the systemic circulation rather than the RES organs.

Comparative Pharmacological Efficacy Study of the Dosage Form (Urine Volume Test)

One of the measurable pharmacological efficacy indicator for Compound 1 is an increase in the urine output. This test for Compound 1 efficacy from different formulations has been extensively used by performing the test in rats.

The head to head comparative study of the two liposomal formulations (Table 10) at 1:5:7 and 1:3:7 Compound 1:EPG:DMPC ratios with the cosolvent formulation was conducted in rats using the DMSO:PEG 200 (50:50) Compound 1 solution as a control. The new liposomal formulations provided 70 to 90% of the urine output and with tighter RSD values as compared to the DMSO:PEG 200 control. The control formulation containing DMSO is generally not acceptable as an intravenous product. The cosolvent formulation was a mixture of Propylene glycol, PEG 400, Ethanol, Benzyl Alcohol and antioxidants and it provided only 50% of the control value for urine output.

TABLE 10
Pharmacological Efficacy of VPA-985 Liposomal
Formulations (By the Rat Urine Output Assay)

| Compound 1 | Urine Output | | Urine (Percent of |
|---|---|---|---|
| Formulations | Average (ml) | RSD | Control) |
| DMSO:PEG 200 | 21.3 | 19.5 | 100.0% |
| (50:50) Control* | | | |
| Cosolvent*+ | 10.7 | 11.8 | 50.0% |
| Liposome (1:5:7 | 14.7 | 9.8 | 68.8% |
| Molar Ratio)# | | | |
| Liposome (1:3:7 | 19.0 | 8.1 | 89.1% |
| Molar Ratio)# | | | |

*Resulted in Hematuria (Blood in Urine), whereas liposomal formulations were free of hematuria
+Cosolvent composition w/v % (Drug 1%:PEG 400 34%:Ethanol 7.9%:Benzyl Alcohol 2.0%:Propylene Glycol 55%:BHT 0.001%)
#Molar ratio as shown (Compound 1:EPG:DMPC)

Pharmacokinetic Study Comparing Cosolvent and Liposomal Dosage Forms:

The summary of the results from the pharmacokinetic study comparing Compound 1 co solvent and 1:3:7 liposomal formulation is provided in Table 11. These data show that the liposomes behave more like solution delivering the HTA in the systemic circulation.

The comparative Intravenous Dose Ranging study was in the ascending dose (3 days/dose) in male dogs. Each formulation was dosed in two dogs for three days at each dose level (0.5, 2.5, and 5.0 mg/kg/day) and blood samples were drawn on 3, 6, and 9 days for Compound 1 analysis.
(1) The pharmacokinetic parameters of Compound 1 in both formulations increase with increasing dose; and may be slightly greater than the proportional dose.
(2) The Cₘₐₓ values at 0.5 and 2.5 mg/kg dose do not appear to be different between the two formulations. At 5.0 mg/kg/day, the liposomal formulation Cₘₐₓ values appears to be higher than those of the co-solvent. Likely factor may be that at higher doses the drug may precipitate from the cosolvent and hence is not available.
(3) The mean AUC₀₋₂₄ values in the cosolvent group are somewhat higher than those in the liposomal group, but a small sample size prohibits a definitive assessment.

The preliminary pharmacological effect test by urine output in rats and the pharmakokinetic studies in dogs indicate that unlike conventional liposomes the new Compound 1 liposomal formulations described act more like solution of the drug in organic solvents and probably delivers the hydrophobic drug prior to reaching liver, to one or more compartments of the blood which in turn become circulatory drug carrier not recognizable by the reticuloendothelial system.

TABLE 11
Toxicokinetic Parameters of Cmpd. 1 in Dogs Following IV Administration of 0.5, 2.5 and 5.0 mg/kg/day
Cmpd. 1 in a Cosolvent or Liposomal Formulation (SN 96242)

| | | Dose | | Cₘₐₓ¹ | | Iₘₐₓ¹ | AUC₀₋₂₄ | | Cl₁ | Vₛₛ | I_{1/2} |
|---|---|---|---|---|---|---|---|---|---|---|---|
| Treatment | Day | (mg/kg/day) | Animal # | (ng/mL) | Cₘₐₓ/Dose | (hr) | (ng * hr/mL) | AUC/Dose | (mL/hr/kg) | (L/kg) | (hr) |
| Cosolvent | 3 | 0.5 | 3 | 351 | 702 | 0.08 | 297² | 595 | 1456 | 1.3 | 0.73 |
| Formulation | | | 4 | 239 | 478 | 0.08 | 1346 | 2692 | 204 | 6.2 | 24 |
| | | | Mean | 295 | 590 | 0.08 | 822 | 1643 | 830 | 3.8 | 12 |
| | | | n | 2 | 2 | 2 | 2 | 2 | 2 | 2 | 2 |
| | 6 | 2.5 | 3 | 1883 | 753 | 0.08 | 3844 | 1538 | 492 | 7.6 | 16 |
| | | | 4 | 991 | 396 | 0.18 | 5774 | 2310 | 296 | 5.9 | 16 |
| | | | Mean | 1437 | 575 | 0.13 | 4809 | 1924 | 394 | 6.7 | 16 |
| | | | n | 2 | 2 | 2 | 2 | 2 | 2 | 2 | 2 |
| | 9 | 5 | 3 | 3705 | 741 | 0.08 | 9168 | 1834 | 486 | 3.8 | 9.4 |
| | | | 4 | 3134 | 627 | 0.08 | 13349 | 2670 | 282 | 4.4 | 14 |
| | | | Mean | 3420 | 684 | 0.08 | 11259 | 2252 | 384 | 4.1 | 12 |
| | | | n | 2 | 2 | 2 | 2 | 2 | 2 | 2 | 2 |
| Liposomal | 3 | 0.5 | 5 | 330 | 660 | 0.10 | 617² | 1234 | 752 | 1.2 | 1.1 |
| Formulation | | | 6 | 318 | 636 | 0.10 | 245² | 490 | 1750 | 1.6 | 0.8 |
| | | | Mean | 324 | 648 | 0.10 | 431 | 862 | 1246 | 1.4 | 1 |
| | | | n | 2 | 2 | 2 | 2 | 2 | 2 | 2 | 2 |
| | 6 | 2.5 | 5 | 1919 | 768 | 0.11 | 4522 | 1809 | 491 | 3.9 | 9.7 |
| | | | 6 | 1902 | 761 | 0.08 | 1716² | 686 | 1267 | 2.1 | 1.9 |
| | | | Mean | 1911 | 764 | 0.095 | 3119 | 1299 | 879 | 3.0 | 6 |
| | | | n | 2 | 2 | 2 | 2 | 2 | 2 | 2 | 2 |
| | 9 | 5 | 5 | 5583 | 1117 | 0.08 | 12871 | 2574 | 321 | 3.6 | 12 |
| | | | 6 | 4738 | 948 | 0.08 | 8358 | 1672 | 555 | 3.3 | 8.1 |
| | | | Mean | 5161 | 1032 | 0.08 | 10615 | 2123 | 438 | 3.5 | 10 |
| | | | n | 2 | 2 | 2 | 2 | 2 | 2 | 2 | 2 |

¹Cₘₐₓ was the highest observed concentration, Imax was the first sampling time, no extrapolation in C₀ was performed.
²AUC₀₋ₜ, as the Cmpd. 1 concentrations at 24 hours in these dogs were <25 ng/mL, and no AUC₀₋₂₄ was calculated.

What is claimed is:

1. A lyophilized liposomal composition comprising
(i) a hydrophobic therapeutic agent;
(ii) a first component; and
(iii) a second component;
wherein, when the composition is contacted with water, the first component and the second component interact to form a substantially homogeneous liposomal solution of the hydrophobic therapeutic agent.

2. The composition of claim 1, wherein the composition comprises from about 20 weight percent to about 40 weight percent of the first component and the second component.

3. The composition of claim 1, wherein the weight per cent ratio of the second component to the first component is from about 1 to about 7.

4. The composition of claim 1, wherein the number of moles of the first component is about the same as the number of moles of the hydrophobic therapeutic agent, and the number of moles of the second component is from about 2 to about 15 times greater than the number of moles of the hydrophobic therapeutic agent.

5. The composition of claim 1, wherein the number of moles of the first component is from about 1.5 to about 6 times greater than the number of moles of the hydrophobic therapeutic agent, and the number of moles of the second component is from about 2 to about 15 times greater than the number of moles of the hydrophobic therapeutic agent.

6. The composition of claim 1, wherein the weight per cent ratio of the first component and the second component to the hydrophobic therapeutic agent is from about 10 to about 50.

7. The composition of claim 1, wherein each of the first component and the second component is, independently, a natural lecithin or phospholipid.

8. The composition of claim 7, wherein the first component is egg phosphatidyl glycerol and the second component is soy phosphatidyl choline.

9. The composition of claim 1, wherein the composition comprises from about 0.05 weight percent to about 10 weight percent of the hydrophobic therapeutic agent.

10. The composition of claim 1, wherein the composition further comprises a cryoprotectant.

11. The composition of claim 10, wherein the cryoprotectant is a sugar.

12. The composition of claim 11, wherein the cryoprotectant is lactose.

13. The composition of claim 1, wherein the composition further comprises an anti-oxidant.

14. The composition of claim 13, wherein the composition comprises two anti-oxidants.

15. The composition of claim 14, wherein the two anti-oxidants are BHT and ascorbyl palmitate.

16. The composition of claim 1, wherein the composition further comprises a cryoprotectant, a first anti-oxidant, and a second anti-oxidant.

17. The composition of claim 16, wherein the composition comprises:

| Ingredient | Amount % (w/w) |
|---|---|
| Hydrophobic therapeutic agent | about 0.500 to about 2.500 |
| First Component | about 5 to about 15 |
| Second Component | about 15 to about 25 |
| First Anti-oxidant | about 0.005 to about 0.020 |
| Second Anti-oxidant | about 0.025 to about 0.050 |
| Cryoprotectant | about 50 to about 75 |

18. The composition of claim 16, wherein the cryoprotectant is lactose, the first anti-oxidant is BHT, and the second anti-oxidant is ascorbyl palmitate.

19. The composition of claim 1, wherein the hydrophobic therapeutic agent has a water solubility of from about 5 nanograms/mL to about 5 milligrams/mL.

20. The composition of claim 19, wherein the hydrophobic therapeutic agent has a molecular weight of from about 100 Daltons to about 1,000 Daltons.

21. The composition of claim 19, wherein the hydrophobic therapeutic agent lacks ionizable groups.

22. The composition of claim 19, wherein the hydrophobic therapeutic agent further comprises an acidic group having a pKa of from about 2 to about 11.

23. The composition of claim 19, wherein the hydrophobic therapeutic agent further comprises a basic group, wherein the pKa of the basic group's conjugate acid is from about 3 to about 12.

24. The composition of claim 19, wherein the hydrophobic therapeutic agent is a zwitterion.

25. The composition of claim 19, wherein the hydrophobic therapeutic agent is a crystalline solid.

26. The composition of claim 19, wherein the hydrophobic therapeutic agent further comprises two rings, wherein each ring is, independently, an aromatic ring or a heteroaromatic ring.

27. The composition of claim 19, wherein the hydrophobic therapeutic agent further comprises a condensed bicyclic, tricyclic or polycyclic ring system.

28. The composition of claim 19, wherein the hydrophobic therapeutic agent is a water insoluble fungal antibiotic or complex macrocycle of synthetic, semi-synthetic, or natural origin.

29. The composition of claims 1 or 19, wherein the hydrophobic therapeutic agent has a log P value of from about 1.0 to about 5.0.

30. The composition of claims 1 or 19, wherein the hydrophobic therapeutic agent has a log P value of from about 2.0 to about 5.0.

31. The composition of claims 1 or 19, wherein the hydrophobic therapeutic agent has a log P value of from about 3.0 to about 5.0.

32. The composition of claims 1 or 19, wherein the hydrophobic therapeutic agent has a log P value of from about 4.0 to about 5.0.

33. A process for preparing a composition of claim 1, the process comprising:
(i) combining a hydrophobic therapeutic agent, a first component, and a second component in an organic solvent to form a first combination;
(ii) combining the first combination with a water phase to form a second combination;
(iii) removing the organic solvent from the second combination to form a third combination; and
(iv) lyophilizing the third combination, thereby preparing the composition of claim 1.

34. The process of claim 33, wherein the organic solvent is ethanol.

35. The process of claim 33, wherein the water phase further comprises a cryoprotectant.

36. The process of claim 35, wherein the cryoprotectant is lactose.

37. The process of claim 33, wherein the first combination further comprises an anti-oxidant.

38. The process of claim 33, wherein the second combination is a liposomal solution.

39. The process of claim 38, wherein the process further comprises the step of reducing the particle size distribution of the liposomes.

40. The process of claim 38, wherein the process further comprises the step of reducing the particle size distribution of the liposomes to a final particle size distribution of from about 5,000 nm to about 20 nm.

41. The process of claim 38, wherein the process further comprises the step of reducing the particle size distribution of the liposomes to about 200 nm.

42. The process of claim 33, wherein step (iii) comprises performing a tangential flow filtration.

43. The process of claim 42, wherein the organic solvent is ethanol.

44. A substantially homogeneous liposomal formulation comprising:
(i) a hydrophobic therapeutic agent;
(ii) a first component;
(iii) a second component; and
(iv) water.

45. The formulation of claim 44, wherein the formulation comprises at least about 80 weight/volume per cent of water.

46. The formulation of claim 44, wherein the formulation further comprises a cryoprotectant, a first anti-oxidant, and a second anti-oxidant.

47. The formulation of claim 46, wherein the formulation comprises:

| Ingredient | Amount % (w/v) |
|---|---|
| Hydrophobic therapeutic agent | about 0.050 to about 0.500 |
| First Component | about 0.5 to about 5.0 |
| Second Component | about 1.5 to about 6.0 |
| First Anti-oxidant | about 0.001 to about 0.005 |
| Second Anti-oxidant | about 0.004 to about 0.008 |
| Cryoprotectant | about 5 to about 15 |
| Water | about 70 to about 90 |

48. The formulation of claim 44, wherein the formulation comprises about 2 mg/mL of the hydrophobic therapeutic agent.

49. The formulation of claim 44, wherein the formulation is an intravenous formulation for administration to a human or animal subject.

50. The formulation of claim 44, wherein the formulation is prepared by contacting the lyophilized lipsomal composition of claim 1 with water.

51. The formulation of claim 44, wherein the liposomes have an average particle size distribution of at most about 5,000 nm.

52. The formulation of claim 44, wherein the liposomes have an average particle size distribution of from about 50 nm to about 200 nm.

53. The formulation of claim 44, wherein the liposomes have an average particle size distribution of about 200 nm.

54. The formulation of claim 44, wherein the formulation is capable of being diluted indefinitely with water without precipitation of the hydrophobic therapeutic agent.

55. The formulation of claim 44, wherein the formulation rapidly releases the hydrophobic therapeutic agent into the bloodstream to associate with red blood cell (RBC), lipoproteins, HSA or WBC in blood upon in vivo administration.

56. The composition of claim 1, wherein the number of moles of the first component is less than the number of moles of the hydrophobic therapeutic agent, and the number of moles of the second component is from about 2 to about 15 times greater than the number of moles of the hydrophobic therapeutic agent.
